(19) United States
(12) Patent Application Publication
(10) Pub. No.: US 2021/0371893 A1
(43) Pub. Date: Dec. 2, 2021

(54) CELL-FREE PROTEIN SYNTHESIS METHOD

(71) Applicant: Jiangsu Zhidian Biotechnology Co., Ltd., Xuzhou (CN)

(72) Inventors: Lei KAI, Xuzhou (CN); Kun HAN, Xuzhou (CN); Jiachang LIAN, Xuzhou (CN); Yi MA, Xuzhou (CN); Ke YUE, Xuzhou (CN)

(73) Assignee: Jiangsu Zhidian Biotechnology Co., Ltd., Xuzhou (CN)

(21) Appl. No.: 17/265,527

(22) PCT Filed: Aug. 7, 2020
(86) PCT No.: PCT/CN2020/107639

§ 371 Date: Feb. 3, 2021

(30) Foreign Application Priority Data
Mar. 18, 2020 (CN) 202010190489.9

(51) Int. Cl. C12P 21/00; C12M 1/32; C12M 1/12

(52) U.S. Cl. CPC C12P 21/00; C12M 25/04; C12M 23/12

(57) ABSTRACT

A cell-free protein synthesis method includes the following steps: (i) providing a multi-well plate, the multi-well plate includes a cover plate and a base provided with a plurality of wells. Each well is formed by one or more side walls, a bottom II and an opening, and the cover plate matches the opening; (ii) providing fluid to some of the wells; (iii) adding a biochemical factor and one or more of a template DNA, a template RNA_{;} an additive, and a reaction cofactor into the fluid; or (iv) adding one or more of the template DNA, the template RNA, the additive, and the reaction cofactor to the fluid; (v) placing the cover plate on a top of the base to seal the openings of the wells; and (vi) subjecting the multi-well plate to incubation for a period of time.

### CROSS REFERENCE TO THE RELATED APPLICATIONS

This application is the national phase entry of International Application No. PCT/CN2020/107639, filed on Aug. 7, 2020, which is based upon and claims priority to Chinese Patent Application No. 202010190489.9, filed on Mar. 18, 2020, the entire contents of which are incorporated herein by reference.

### TECHNICAL FIELD

The present invention relates to the technical field of biotechnology, and particularly to a cell-free protein synthesis method.

### BACKGROUND

Cell-free protein synthesis (CFPS) is also known as in vitro protein synthesis. The purpose of this process is to produce proteins based on cellular biological mechanisms without being restricted to living cells. As long as the concentrations of reaction components are sufficient, the cell-free protein synthesis process can produce proteins sustainably. In general, cell-free protein synthesis requires the presence of amino acids, DNA or RNA templates that encode the desired proteins, ribosomes, tRNAs, and energy sources. Moreover, cell-free protein synthesis can be performed with purified individual components or cell extracts.

In the field of in vitro biological experiments, such as cell-free protein synthesis and fluorescence assays, screening reactions are usually carried out in standard well plates, including 24-well, 48-well, 96-well, 384-well, 1024-well or other customized well plates. Although these plates are widely used, they have the following disadvantages for use in the field of in vitro biological experiments: the volume provided by each well in the above-mentioned standard well plates is relatively large, for example, in a standard 96-well plate, a volume of approximately 360 μL is provided by each well. Generally, the working volume used in each well ranges from hundreds of microliters to several milliliters. For a 96-well plate with all wells for the above reaction, the cost of reagents can quickly rise to thousands of dollars, resulting in high usage costs.

### SUMMARY

The objective of the present invention is to provide a cell-free protein synthesis method, which provides an improvement over the synthesis method known in the art, thereby reducing the reagent cost.

To achieve the objective of the present invention, the present invention provides a cell-free protein synthesis method, which includes the following steps:

a. providing a multi-well plate, in which the multi-well plate includes a base and a cover plate. The base is provided with a plurality of wells. Each well is formed by one or more side walls. A bottom Il and an opening, and the cover plate matches the opening. A volume of a reaction cavity of each well is less than 20 μL, and some of the wells in the plurality of wells communicate with each other;

b. providing a certain volume of fluid to some of the wells in the plurality of wells in step a, in which the fluid is a cell-free reaction mixture or the fluid is a mixture of the cell-free reaction mixture and a biochemical factor;

c. when the fluid in step b is the cell-free reaction mixture, adding the biochemical factor and at least one selected from the group consisting of a template DNA, a template RNA, an additive, and a reaction cofactor into the wells where the fluid is added in step b. When the fluid in step b is the mixture of the cell-free reaction mixture and the biochemical factor, adding at least one selected from the group consisting of the template DNA, the template RNA, the additive, and the reaction cofactor to the wells where the fluid is added in step b;

d. placing the cover plate on a top of the base to close the openings of the wells, and the fluid added in step b is in contact with the bottom II of each well and the cover plate; and

e. subjecting the multi-well plate of step d to an incubation for a period of time under suitable conditions.

Preferably, the volume of the reaction cavity of each well is less than 10 μL; preferably, the volume of the reaction cavity of each well is less than 5 μL; preferably, the volume of the reaction cavity of each well is less than 3 μL. By reducing the height of the well, the volume of the reaction cavity can be reduced, and a smaller volume of the liquid can be used in the reaction cavity, thereby reducing the reagent cost.

The cell-free protein synthesis method provided by the present invention requires less reagent because the multi-well plate used therein has a smaller well volume. In addition, the reaction fluid is in contact with the bottom II and the cover plate simultaneously, so that the evaporation of the liquid can be greatly reduced. This is extremely beneficial to the processing of trace liquids. In addition, when the cover plate is placed on the base, an airtight seal can be formed on the opening of each well, and the airtight seal reduces and/or prevents the evaporation of fluid from the wells, Preventing evaporation loss ensures that the biochemical concentration within the fluid volume remains at the desired level, which will not change over time.

Preferably, in the method, when one or more biochemical factors are introduced into the wells of the multi-well plate in step b or step c, amounts or concentrations of the biochemical factors form an incremental gradient between the plurality of wells. When the fluid in step b is the mixture of the cell-free reaction mixture and the biochemical factors, an optical measurement experiment can be quickly performed by pre-mixing the biochemical factors.

Preferably, the wells of the multi-well plate are positioned in a matrix form. When two biochemical factors are provided, a first biochemical factor forms an incremental gradient between a first gradient of the matrix, and a second biochemical factor forms an incremental gradient between a second gradient of the matrix. That is, when two biochemical factors are provided, the first biochemical factor forms the incremental gradient between a first row of the matrix, and the second biochemical factor forms the incremental gradient between a first column of the matrix; that is, when two biochemical factors are provided, the first biochemical factor forms the incremental gradient along a length direction of the multi-well plate, and the second biochemical factor forms the incremental gradient along a width direction of the multi-well plate.

Preferably, the biochemical factor in step b or step c is one or more selected from M a nucleoside triphosphate (NTP) mixture, an amino acid mixture, and an energy mixture.

Preferably, the method further includes the steps of: after introducing the fluid into at least some of the wells, freeze-drying the fluid, and hydrating the freeze-dried fluid by providing water thereto. When the fluid in step b is the mixture of the cell-free reaction mixture and the biochemical factor, such assays can be pipelined and simplified for the user by providing the fluid with the biochemical factor that has been freeze-dried in the wells of the multi-well plate.

Preferably, either or both of the bottom II and the cover plate are transparent. Providing a multi-well plate that is transparent on at least one side enables imaging of the reaction product without removing the cover plate of the multi-well plate. Preferably, one or both of the bottom Il and the cover plate are at least partially made of a glass or a plastic. Preferably, one or both of the bottom 11 and the cover plate are at least partially made of any one or both of a copolymer of polypropylene and cycloolefin, and polystyrene.

Preferably, the base further includes a spacer forming the one or more side walls of the plurality of wells. A cover-facing side of the spacer is coated with or composed of an adhesive material. Adhesive attachment can further facilitate the user's operation of the multi-well plate, especially when the fluid movement in the well is reduced through contact the fluid with the bottom II of the well and the cover plate. The cover-facing side of the spacer is further provided with a protective film. Providing the protective film helps to protect the adhesive coating on the base until the base and the cover plate are sealed together in an airtight mariner, which further facilitates the use of the multi-well plate in the laboratory.

### BRIEF DESCRIPTION OF THE DRAWINGS

FIG. 1 is a vertical cross-sectional view of an existing reaction well for cell-free protein synthesis;

FIG. 2A is a cross-sectional view of a single well in a multi-well plate of the present invention with a deposited fluid but no cover plate;

FIG. 2B is a cross-sectional view of a single well in a multi-well plate of the present invention with a deposited fluid and the cover plate fixed in place; and

FIG. 3 is a top view with a concentration gradient observed from above the multi-well plate of the present invention.

In the drawings:

1. reaction well; 10. bottom 1; 20. well cavity; 30. surrounding partition; 70. solution; 100. well; 110. base; 120. reaction cavity; 130. side wall; 140; bottom II; 150. opening; 160. cover plate; 170. fluid; 200. multi-well plate; 210. first gradient; 220. second gradient; and 230. dialysis membrane.

### DETAILED DESCRIPTION OF THE EMBODIMENTS

The present invention is further described in detail hereinafter with reference to the embodiments and the drawings.

As described in this application, the term “protein synthesis” refers to the assembly of proteins from amino acids. The plate or multi-well plate as described in this application refers to a vessel or container used for biological or chemical analysis. The term “plate” shall not be construed as limitations to the size, structure or material of the plate.

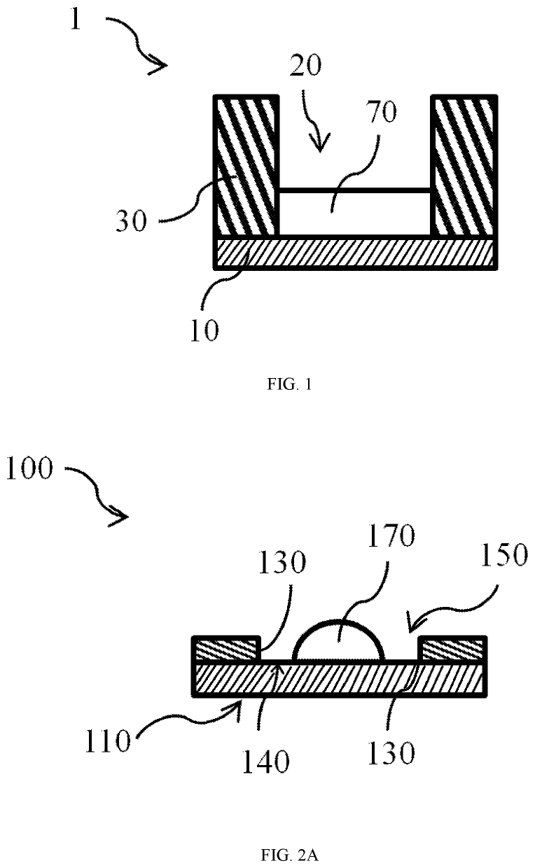

FIG. 1 shows the existing reaction well 1 for cell-free protein synthesis. The reaction well 1 is provided with the bottom I 10 and the surrounding partition 30 for forming the well cavity 20. The well cavity 20 in the well 1 of the prior art is relatively large, usually larger than 200 μL. Therefore, when the solution 70 is deposited in the reaction well 1, the volume of the solution 70 must be large enough (usually greater than 20 μL) to allow sufficient experiments.

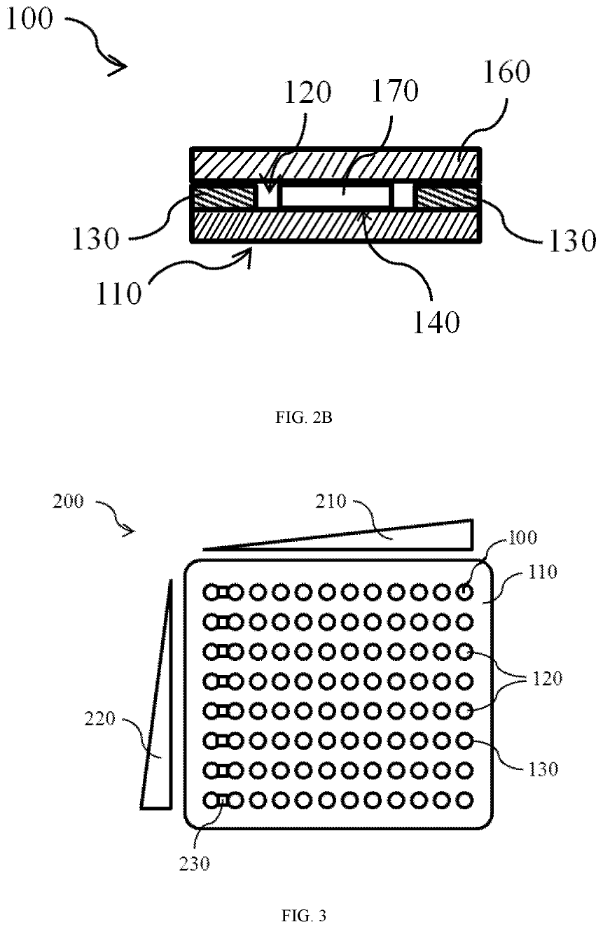

FIGS. 2A and 2B both provide a cross-sectional view of the well 100 of the multi-well plate of the present invention. The multi-well plate 200 includes the base 110 provided with a plurality of wells 100. Each well 100 provides the reaction cavity 120, and each well 100 includes at least one side wall 130. Each well 100 further includes the opening 150 at a top of the well 100 and the bottom II 140. FIGS. 2A and 2B further show a certain volume of the fluid 170 deposited in well 100. As shown in FIG. 2B, the well 100 has the cover plate 160 provided at the top of the well 100.

The base 110 of the multi-well plate 200 is provided with a plurality of wells 100, and each well 100 is formed by one or more side walls 130, the bottom II 140 and the opening 150. The bottom II 140 can be made of a glass or a plastic, such as polypropylene and polystyrene, a copolymer of the polypropylene and cycloolefin, and a copolymer of the polypropylene, the polystyrene and the cycloolefin. Preferably, the bottom II 140 is at least partially transparent, for example, the bottom II 140 is transparent at least at certain wavelengths. The transparent bottom II 140 can realize the imaging of the contents in the well 100 from below (such as using an inverted microscope) without interfering with the contents of the well 100. The width of the bottom II 140 may depend on the requirements of the detection performed and the type of imaging performed.

The single side wall 130 and the bottom II 140 may form a cylindrical shape. The well 100 may further include a plurality of side walls 130, and the plurality of side walls 130 form a square well when viewed from above, or form other polygonal shapes when viewed from above. One or more side walls 130 may also be made of a glass or a plastic (such as polypropylene and polystyrene, a copolymer of the polypropylene and cycloolefin, and a copolymer of the polypropylene, the polystyrene and the cycloolefin), and may have the same characteristics, and/or be formed integrally with the bottom II 140. However, in some configurations, the plurality of side walls 130 may be made of other different materials, such as adhesive materials, so that the opening 150 can be better sealed by the cover plate 160. One or more side walls 130 may further be made of partially opaque and/or dark-colored materials, which may help to visually distinguish the wells in the imaging configuration. In order to contain only a small amount of the fluid 170, the side wall 130 may have a low height. This height can provide a well depth of less than 1 mm, preferably less than 0.5 ram, or more preferably less than 0.2 mm. When one or more side walls 130 are made of the adhesive material, it is preferable to form a low-height structure. When the side wall 130 is not made of the adhesive materials, it is difficult to form the closed reaction cavity 120 between the side wall 13( )and the cover plate 160, resulting in the fluid 170 escaping between the well 100 and the cover plate 160. The side wall 130 can also be in the form of a spacer that not only forms the wall of well 100, but also fills the entire space between the wells 100 on the multi-well plate 200, The side wall 130 or the cover-facing side of the spacer is composed of or coated with an adhesive material, which helps to seal the well 100 against the cover plate 160, thereby isolating the contents of the well 100 from the surrounding environment.

The height of one or more side walls 130 and the surface area of the bottom II 140 occupied by the well 100 together define the volume of the well 100. The volume of the well 100 is small in order to contain a small amount of the fluid 170 without exposing the fluid 170 to a large amount of surrounding air. The volume of each well of the multi-well plate in some existing configurations is relatively large, such as 50 μL, 200 μL, or even as high as 1,000 μL, while the volume of each well of the multi-well plate of the present invention is less than 20 μL, preferably less than 10 μL, more preferably less than 5 μL, and more preferably less than 3 μL, depending on the specific application.

The cover plate 160 may be made of a glass or a plastic, for example, made of any one or both of a copolymer of polypropylene and cycloolefin, and polystyrene. Preferably, the cover plate 160 may be transparent at least at certain wavelengths of light, which enables imaging of the contents in the well 100 from above without interfering with the contents of the well 100.

An advantageous configuration of the multi-well plate 200 is in which an airtight seal is formed on the opening 150 of the well 100 when the cover plate 160 is closed in place. The airtight seal can prevent liquid from evaporating and losing from the well 100. Since the evaporation loss of the liquid over time may make the concentration within the reaction well 100 unreliable, the prevention of the evaporation can yield more reliable results from the detection performed in the well 100.

The well 100 of the multi-well plate 200 can optionally be coated with a sealing liquid such as bovine serum albumin (BSA), polyethylene glycol (PEG) and/or silane on the inner wall(s) and the bottom II 140 of the well 100 before use, which ensures that the bottom II 140 and the side walls 130 are coated with a non-reactive coating to minimize non-specific binding effects.

As shown in FIG. 3, some wells in the plurality of wells 100 are intercommunicated with each other. Among the intercommunicated wells, one is set as the main well and another one as the side well. The main well and the side well are set artificially. In an advantageous configuration, the multi-well plate 200 may further include one or more dialysis membranes 230, and these dialysis membranes 230 are arranged between the intercommunicated wells 100. The fluid 170 contained in the main well is in contact with another fluid 170 containing a certain concentration of biochemical factor contained in the side well. The slow dialysis of the biochemical factors through the dialysis membrane 230 allows the biochemical reaction to continue in a longer period of time, that is, to extend the reaction time while maintaining the concentration of the fluid 170 at an optimal level.

Referring to FIGS. 2A, 2B and 3, the present invention provides a cell-free protein synthesis method.

First, the multi-well plate 200 is provided. The multi-well plate 200 includes the base 110 and the cover plate 160. The base 110 is provided with the plurality of wells 100, as shown in FIGS. 2A and 213. The cover plate 160 matches the opening 150 and the cover plate 160 is placed on the top of the base 110 to close the opening 150 of the well, thereby completely sealing the well 100 from the external environment. Each of the wells 100 has a small volume, and a maximum volume of the reaction cavity 120 of each well is 20 μL, preferably 10 μL, more preferably 5 μL, and more preferably less than 3 L. As shown in FIG. 2A, a certain volume of the fluid 170 is deposited in at least one well 100 of the multi-well plate 200, which can be achieved by manual pipetting or by automatic pipetting or by an automatic liquid handling system. In some configurations of the method, an additional microfluidic system can be configured to deposit a certain volume of the fluid 170 within the well 100.

The fluid 170 with this volume of the present invention includes a cell-free reaction mixture. The cell-free reaction mixture includes a plurality of components, The cell-free reaction mixture may include a base solution such as water, salt solution, or a commercially available buffer that provides other factor suspension for the cell-free reaction mixture. The cell-free reaction mixture further includes energy sources, such as glucose or ATP (Adenosine Triphosphate), amino acid mixtures, kinases or other enzymes, salts, pH buffers, or other biological factors and/or chemical factors. Further, the cell-free reaction mixture includes ribosomes used for protein synthesis from amino acids and/or tRNA to complete the assembly of amino acids.

Before or after introducing a certain volume of fluid, the user introduces the biochemical factors required for the initiation of the reaction. In the case of cell-free protein synthesis, the liquid with this volume of the present invention may include template DNAs, template RNAs, additives, and/or reaction cofactors.

Once all the necessary components are introduced into the well 100, the biochemical process begins. Then, as shown in FIG. 2B, the user closes the cover plate 160 to seal the single well 100. One or more side walls 130 and the bottom II 140 of the base 110 together with the cover plate 160 form an enclosed chamber with a cell-free reaction mixture inside. Since the volumes of the well 100 and the fluid 170 are all small, the fluid 170 is in contact with both the bottom I1 140 of the well 100 and the cover plate 160, thus the fluid 170 becomes a slightly flat disc shape. In some configurations, the fluid 170 may further contact one or more side walls 130 of the well 100. Since the volume of each well 100 is 20 μL, or less, preferably 10 μL, more preferably 5 μL, and more preferably 3 μL, or less, the volume of the fluid 170 to be used in the well 100 must be much smaller. For example, in a 10 μL well, the fluid 170 with a volume of 9 μL can be used. Since the volume of the fluid 170 in the well 100 is significantly reduced, the cost of the reagents can be reduced. In addition, in the closed state, the volume of the fluid 170 in contact with air is much smaller, so that the evaporation of the fluid 170 is significantly reduced, thereby ensuring that the concentrations of reagents and products in the well 100 are maintained at an optimal level during the detection period.

Finally, the covered multi-well plate 200 is incubated for a certain period of time so that the protein synthesis reaction can proceed. Incubation generally refers to providing the required environmental conditions that promote the reactions for a given assay. Incubation may include keeping the wells 100 of the multi-well plate 200 at a given temperature of 20° C.-40° C. Incubation can further include providing some type of air, such as purified and/or humidified air. The incubation time can be minutes, hours or even days, depending on the type of reaction and the requirements of the assay.

In a preferred embodiment of the method, at least one biochemical factor is introduced into the plurality of wells 100 of the multi-well plate 200, so that one or more biochemical factors form an incremental gradient between the plurality of wells 100. Preferably, the increase in the amount and/or concentration of the one or more biochemical factors follows a predetermined. function, preferably a linear function. However, logarithmic or exponential functions can also be used. When more than one biochemical factor is introduced, different biochemical factors are introduced by following different functions of the amount or concentration between the wells. For example, different linear functions, linear and logarithmic functions, linear and exponential functions, and others.

For example, the wells of the multi-well plate may be formed in one column, one row, one column and one row, one column and a plurality of rows, a plurality of columns and one row, a plurality of columns and a plurality of rows. When the first biochemical factor is used, the first biochemical factor may be provided with an incremental gradient along one column, the first column of the plurality of columns, one row, or the first row of the plurality of rows, that is, the concentration is gradually changed; and when the second biochemical factor is used, the second biochemical factor may be provided with an incremental gradient along one row, another row in the plurality of rows, one column, or another column in the plurality of columns, that is, the concentration is gradually changed. In other words, the first biochemical factor may be provided with the incremental gradient along one row or the plurality of rows, and the second biochemical factor may be provided with the incremental gradient along one column or the plurality of columns: or, the first biochemical factor can be provided with the incremental gradient along one column or the plurality of columns, and the second biochemical factor can be provided with the incremental gradient along one row or the plurality of rows.

That is, the first biochemical factor may be provided with the incremental gradient along the length direction of the multi-well plate 200, and the second biochemical factor may be provided with the incremental gradient along the width direction of the multi-well plate 200 or the first biochemical factor may be provided with the incremental gradient along the width direction of the multi-well plate 200, and the second biochemical factor may be provided with the incremental gradient along the length direction of the multi-well plate 200. When both biochemical factors are provided in the form of gradients, the gradients can be oriented in different directions (depending on the arrangement of the wells, such as perpendicular to each other), thereby forming a matrix composed of different biochemical factors. Such a configuration is shown in FIG. 3, where the first gradient 210 is formed along the horizontal direction of the wells 100, as symbolically indicated by the gradient bar. The second biochemical factor is deposited as the second gradient 220 along the vertical direction of the wells 100, as shown by the gradient bar. In this way, the gradients of the two biochemical factors form the matrix for the detection experiment, where the top left well (as shown in FIG. 3) contains the smallest amount of two biochemical factors, and the bottom right well contains the largest amount of two biochemical factors. These biochemical factors are frequently used for preliminary reaction screening. The combination of these biochemical factors includes: Mg²⁺ as the first biochemical factor and K⁺ as the second biochemical factor, the Mg²⁺ as the first biochemical factor and a NTP mixture as the second biochemical factor, the Mg²⁺ as the first biochemical factor and an amino acid mixture as the second biochemical factor, the Mg²⁺ as the first biochemical factor and an energy mixture as the second biochemical factor, the K³⁰ as the first biochemical factor and the NTP mixture as the second biochemical factor, the K⁺ as the first biochemical factor and the amino acid mixture as the second biochemical factor, the K⁺ as the first biochemical factor and the energy mixture as the second biochemical factor. the NTP mixture as the first biochemical factor and the amino acid mixture as the second biochemical factor, the⁻N′I′P mixture as the first biochemical factor and the energy mixture as the second biochemical factor, and the amino acid mixture as the first biochemical factor and the energy mixture as the second biochemical factor. Once the detection is performed, the user can easily determine which combination of biochemical factors is most appropriate (for example, which combination provides the highest yield).

The biochemical factor can be any one of the above-mentioned biological or chemical species. The biochemical factor can be Mg²⁺, K⁺, template DNAs, or template RNAs. Preferably, when the well 100 is provided to the user, the biochemical factor is already included in the well 100. For example, the multi-well plate 200 may be provided with a certain volume of the fluid 170 in the wells 100. This configuration of the multi-well plate 200 is advantageous for the user because concentration screening can be performed to obtain the best reaction results. Preferably, for example, when the multi-well plate 200 is provided to the consumer, the biochemical factors have been freeze-dried in the wells 100. Therefore, the multi-well plate 200 can be stored and transported together with freeze-dried biochemical factors already present in the wells in a gradient form, which can realize faster and more simplified concentration screening assays for users.

What is claimed is:

1. A cell-free protein synthesis method, comprising the following steps:
a. providing a multi-well plate, wherein the multi-well plate comprises a base and a cover plate, the base is provided with a plurality of wells, each well of the plurality of wells is formed by at least one side wall, a bottom II and an opening, and the cover plate matches the opening, a volume of a reaction cavity of the each well is less than 20 μL; a predetermined amount of wells in the plurality of wells communicate with each other; the multi-well plate further comprises at least one dialysis membrane, and the at least one dialysis membrane is arranged between the predetermined amount of wells in the plurality of wells;
b. providing a predetermined volume of a fluid to the predetermined amount of wells in the plurality of wells in step a, wherein the fluid is a cell-free reaction mixture, or the fluid is a mixture of the cell-free reaction mixture and at least one biochemical factor;
c. when the fluid in step b is the cell-free reaction mixture, adding the at least one biochemical factor and at least one selected from the group consisting of a template DNA, a template RNA, an additive, and a reaction cofactor into the predetermined amount of wells where the fluid is added in step b; when the fluid in step b is the mixture of the cell-free reaction mixture and the at least one biochemical factor, adding at least one selected from the group consisting of the template DNA, the template RNA, the additive, and the reaction cofactor to the predetermined amount of wells where the fluid is added in step b;
d. placing the cover plate on a top of the base to close the opening of the each well, and the fluid in step b is in contact with the bottom II of the each well and the cover plate; and
e. subjecting the multi-well plate of step d to an incubation.

2. The cell-free protein synthesis method according to claim 1, wherein the volume of the reaction cavity of the each well is less than 10 μL.

3. The cell-free protein synthesis method according to claim 1, wherein when the at least one biochemical factors is introduced in step b or step c, amounts or concentrations of the at least one biochemical factor form an incremental gradient between the plurality of wells.

4. The cell-free protein synthesis method according to claim 3, wherein the plurality of wells of the multi-well plate are positioned in a matrix; when a number of the at least one biochemical factor is two, a first biochemical factor of the at least one biochemical factor forms a first incremental gradient between a first gradient of the matrix, and a second biochemical factor of the at least one biochemical factor forms a second incremental gradient between a second gradient of the matrix; wherein when the number of the at least one biochemical factor is two, the first biochemical factor forms the first incremental gradient between a first row of the matrix, and the second biochemical factor forms the second incremental gradient between a first column of the matrix; wherein when the number of the at least one biochemical factor is two, the first biochemical factor forms the first incremental gradient along a length direction of the multi-well plate, and the second biochemical factor forms the second incremental gradient along a width direction of the multi-well plate.

5. The cell-free protein synthesis method according to claim 1, wherein the at least one biochemical factor in step b or step c is at least one selected from the group consisting of Mg²⁺, K⁺, a NTP mixture, and an amino acid mixture.

6. The cell-free protein synthesis method according to claim 1, further comprising the steps of: freeze-drying the predetermined amount of wells to obtain a freeze-dried fluid, wherein the predetermined amount of wells are added with the fluid in step b, and hydrating the freeze-dried fluid by providing water.

7. The cell-free protein synthesis method according to claim 1, wherein at least one of the bottom II and the cover plate is transparent.

8. The cell-free protein synthesis method according to claim 7, wherein at least one of the bottom II and the cover plate is at least partially made of a glass or a plastic.

9. The cell-free protein synthesis method according to claim 8, wherein at least one of the bottom II and the cover plate is at least partially made of any one or both at least one selected from the group consisting of a copolymer of polypropylene and cycloolefin, and polystyrene.

10. The cell-free protein synthesis method according to claim 1, wherein the base further comprises a spacer, and the spacer forms the at least one side wall of the plurality of wells; a cover-facing side of the spacer is coated with or composed of an adhesive material, and a protective film is further arranged above the cover-facing side.

11. The cell-free protein synthesis method according to claim 2, wherein the volume of the reaction cavity of the each well is less than 5 μL.

12. The cell-free protein synthesis method according to claim 11, wherein the volume of the reaction cavity of the each well is less than 3 μL.

13. The cell-free protein synthesis method according to claim 2, wherein when the at least one biochemical factor is introduced in step b or step c, amounts or concentrations of the at least one biochemical factor form an incremental gradient between the plurality of wells.

14. The cell-free protein synthesis method according to claim 2, wherein the at least one biochemical factor in step b or step c is at least one selected from the group consisting of Mg²⁺, K⁺, a NTP mixture, and an amino acid mixture.

15. The cell-free protein synthesis method according to claim 2, further comprising the steps of: freeze-drying the predetermined amount of wells to obtain a freeze-dried fluid, wherein the predetermined amount of wells are added with the fluid in step b, and hydrating the freeze-dried fluid by providing water.

16. The cell-free protein synthesis method according to claim 2, wherein at least one of the bottom II and the cover plate is transparent.

17. The cell-free protein synthesis method according to claim 2, wherein the base further comprises a spacer, and the spacer forms the at least one side wall of the plurality of wells; a cover-facing side of the spacer is coated with or composed of an adhesive material, and a protective film is further arranged above the cover-facing side.
